(19) United States
(12) Patent Application Publication
(10) Pub. No.: US 2024/0299033 A1
(43) Pub. Date: Sep. 12, 2024

(54) FIBROUS CONNECTIVE TISSUE STABILIZATION APPARATUS

(71) Applicant: Daisy E. Fortkort, Indianapolis, IN (US)

(72) Inventor: Daisy E. Fortkort, Indianapolis, IN (US)

(21) Appl. No.: 18/600,643

(22) Filed: Mar. 8, 2024

Related U.S. Application Data
(60) Provisional application No. 63/450,959, filed on Mar. 8, 2023.

(51) Int. Cl. A61B 17/11; A61B 17/00; A61F 2/00; A61F 2/08

(52) U.S. Cl. CPC A61B 17/1146; A61F 2/08; A61B 2017/00004; A61F 2002/0068; A61F 2220/0016; A61F 2220/0075; A61F 2250/001; A61F 2250/0067

(57) ABSTRACT

A method for repairing a torn portion of fibrous connective tissue including placing the torn portion of the fibrous connective tissue within a tubular construct consisting of a biocompatible, surgically implantable material, wherein the tubular construct is equipped with at least one barbed drawstring having a plurality of barbs on a surface thereof, and wherein the drawstring transforms the tubular construct from a first configuration in which the tubular construct has a first minimum diameter, to a second configuration in which the tubular construct has a second minimum diameter that is less than the first minimum diameter; and using the drawstring to transform the tubular construct into the second configuration; wherein, when the tubular construct is in the second configuration, the plurality of barbs engage the fibrous connective tissue.

### CROSS-REFERENCE TO RELATED APPLICATIONS

This application claims the benefit of priority from U.S. Provisional Application No. 63/450,959, filed Mar. 8, 2023, having the same inventors, and the same title, and which is incorporated herein by reference in its entirety.

### FIELD OF THE DISCLOSURE

The present disclosure relates generally to surgically implantable devices, and more particularly to a fibrous connective tissue stabilization apparatus.

### BACKGROUND OF THE DISCLOSURE

Various devices are known to the art for repairing torn tendons and ligaments. For example, U.S. 2013/0013065 (Bills) discloses a surgical device for assisting in the repair and rehabilitation of a tendon. The device disclosed therein includes a cylindrical, helically wound braid which is similar to a “Chinese finger trap”. The device may be implantable, and may be configured to enclose the repair site of the severed tendon. As tension or pull is applied to the tendon, the device tightens to secure the repair site. The device may improve mobility and use of the tendon by providing stability to the repair site, improving glide through the tendon sheath, allowing diffusion of nutrients to the repair site, and/or reducing adhesions within the tendon sheath.

Other devices of this type are known to the art. See, e.g., Chung et al. [Chung, Nam-Soo & Cho, Jae-Ho & Han, Kyeong-Jin & Han, Seung-Hwan & Lee, Doohyung. (2012). Tendon Trap Technique for Rotator Cuff Repair. Orthopedics. 35. 1035-1038. 10.3928/01477447-20121120-04], which discloses a type of fixation that mimics the mechanism of the Chinese finger trap. The devised knot-free tendon trap technique resembles the double-pulley-suture bridge technique, but all suture limbs share the tension evenly and respond synchronized to the cuff tendon. The technique provides simple biologic repairs of rotator cuff tears.

U.S. Pat. No. 9,550,053 (Ross) discloses a device and method for delivering a high viscosity composition. The composition includes a bioactive agent for delivery to a subject in need thereof, purportedly with high bioavailability and with little loss of the bioactive agent to the natural defense mechanisms of the body. The device includes one or more microneedles with structures fabricated on a surface of the microneedles to form a nanotopography. A random or non-random pattern of structures may be fabricated, such as a complex pattern including structures of differing sizes and/or shapes.

### BRIEF DESCRIPTION OF THE DRAWINGS

FIG. 1 is a perspective view (with a magnified view of a portion thereof) of a first embodiment of a fibrous connective tissue stabilization apparatus in accordance with the teachings herein.

FIG. 2 is a perspective view of the fibrous connective tissue stabilization apparatus of FIG. 1.

FIG. 3 is a side view, partially in section, showing the positioning of torn fibrous connective tissue in the fibrous connective tissue stabilization apparatus of FIG. 1.

FIG. 4 is a perspective view of the fibrous connective tissue stabilization apparatus of FIG. 1.

FIGS. 5-6 are a perspective view and a top view, respectively, of a second embodiment of a fibrous connective tissue stabilization apparatus in accordance with the teachings herein.

### SUMMARY OF THE DISCLOSURE

In one aspect a method for repairing a torn portion of fibrous connective tissue is provided which comprises placing the torn portion of the fibrous connective tissue within a tubular construct consisting of a biocompatible, surgically implantable material, wherein the tubular construct is equipped with at least one barbed drawstring having a plurality of barbs on a surface thereof, and wherein the drawstring transforms the tubular construct from a first configuration in which the tubular construct has a first minimum diameter, to a second configuration in which the tubular construct has a second minimum diameter that is less than the first minimum diameter; and using the drawstring to transform the tubular construct into the second configuration wherein, when the tubular construct is in the second configuration, the plurality of barbs engage the fibrous connective tissue.

In another aspect a device is provided which comprises a surgically repaired fibrous connective tissue, comprising of first and second portions of torn fibrous connective tissue; and a tubular construct encompassing torn portions of first and second portions of sed torn fibrous connective tissue, sed tubular construct consisting of a biocompatible, surgically implantable material, wherein the tubular construct is equipped with at least one barbed drawstring having a plurality of barbs on a surface thereof that engage the first and second portions of sed torn fibrous connective tissue.

### DETAILED DESCRIPTION

While the devices and methodologies disclosed in the prior art for repairing torn tendons and ligaments may have some useful attributes, they also suffer from some infirmities. In particular, while the aforementioned prior art devices disclose the use of mechanisms resembling Chinese finger traps for securing the frayed ends of a torn tendon or ligament, they lack a mechanism to prevent these frayed ends from slipping after these ends have been disposed within the mechanism. This may lead to a failure of the frayed tendon or ligament to regrow in the proper orientation so as to realize a properly repaired and functional tendon or ligament. Moreover, while these devices may allow for the use of various growth hormones, embryonic cells or other stimulants to facilitate the regrowth of the frayed tendon or ligament, they rely on normal diffusion processes from the environment surrounding the repaired tissue. This environment may be subject to disruption or trauma. In addition, these constructs rely on consistent application of tension to the frayed ends of the tendon or ligament to retain them within the finger trap type construct. However, such consistent application of tension may not always be present, and may thus result in slippage of the frayed tendon or ligament from the construct.

It has now been found that the foregoing infirmities may be overcome with the devices and methodologies disclosed herein. In a preferred embodiment, an enclosure for frayed connective tissue, such as tendons or ligaments, is provided. The enclosure consists of a mesh which stabilizes the frayed connective tissue while allowing the tissue to be exposed to various growth hormones, embryonic cells or other stimulants to facilitate the regrowth thereof. Preferably, the mesh comprises one or more biocompatible, surgically implantable materials, and is equipped with at least one barbed, drawstring enclosure. The barbs on this enclosure prevent slippage of the device after implantation. Moreover, in some embodiments, these barbs may be utilized to inject one or more pharmaceutically active materials into the locus of the tear in the connective tissue, thus accelerating the repair thereof.

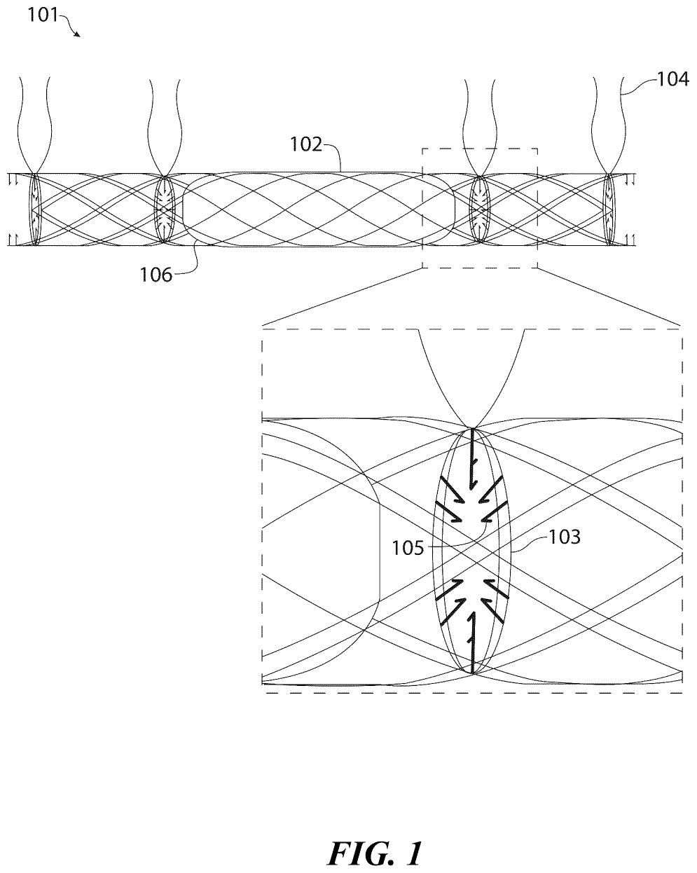

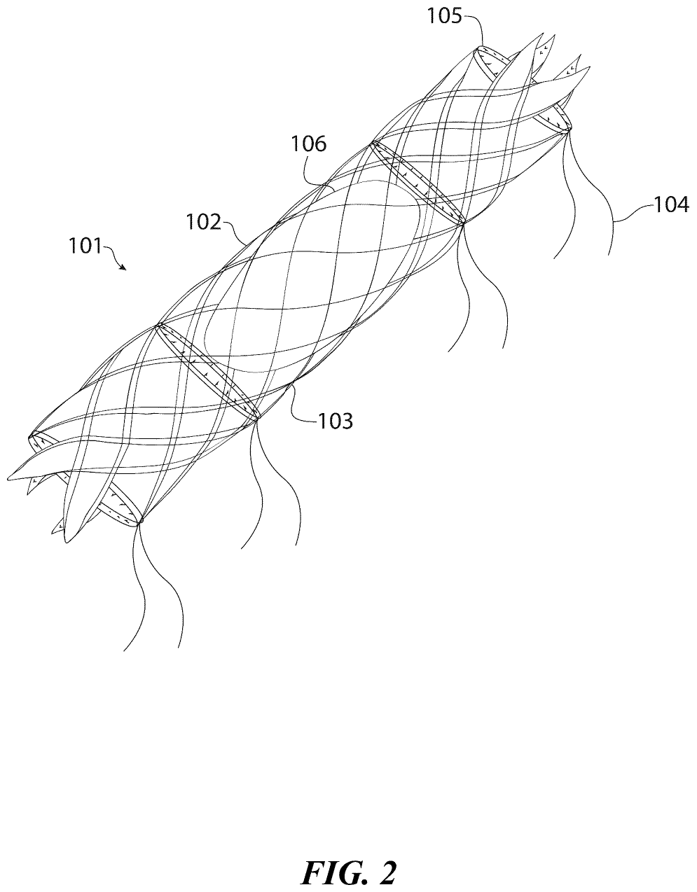

FIGS. 1-2 depict a first particular, non-limiting embodiment of a fibrous connective tissue stabilization apparatus in accordance with the teachings herein. With reference thereto, the fibrous connective tissue stabilization apparatus 101 includes a fibrous, tubular body 102 with a plurality of synchs 103 disposed therein. Each of the plurality of synchs 103 is equipped with external threads 104 which may be utilized to tighten the synchs 103 and reduce the diameter thereof. As best seen in FIG. 1 and the magnified portion thereof, each of the plurality of synchs 103 is equipped with a plurality of inward facing barbs 105.

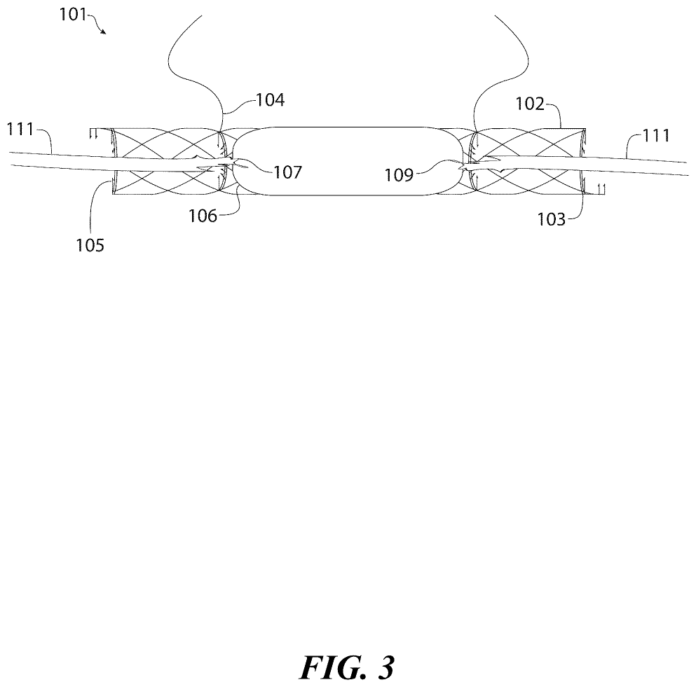

FIG. 3 depicts the use of the fibrous connective tissue stabilization apparatus 101 of FIGS. 1-2 in repairing a torn or frayed ligament, tendon or other fibrous connective tissue. With reference thereto, the frayed portions 107, 109 of the frayed connective tissue 111 are disposed within the fibrous connective tissue stabilization apparatus 101. The external threads 104 are then utilized to tighten the synchs 103. The operating surgeon may then inject various fluids into the mesh of the fibrous connective tissue stabilization apparatus 101. These fluids may include, for example, growth hormones, embryonic cells, or other suitable, pharmaceutically effective materials as may aid in or enhance.

In some variations of the forgoing embodiment, the barbs 105 may be hollow and may be treated or charged with one or more pharmaceutically effective materials as may aid in or enhance the regrowth of the frayed connective tissue. Consequently, as the synchs 103 are tightened these barbs 105 engage the frayed connective tissue and provide for localized injection of the pharmaceutically effective materials therein. This ensures adequate exposure of the frayed connective tissues to the pharmaceutically effective materials, and also prevents slippage of the frayed portions 107, 109 of the connective tissue 111 from the Fibrous Connective Tissue Stabilization Apparatus 101.

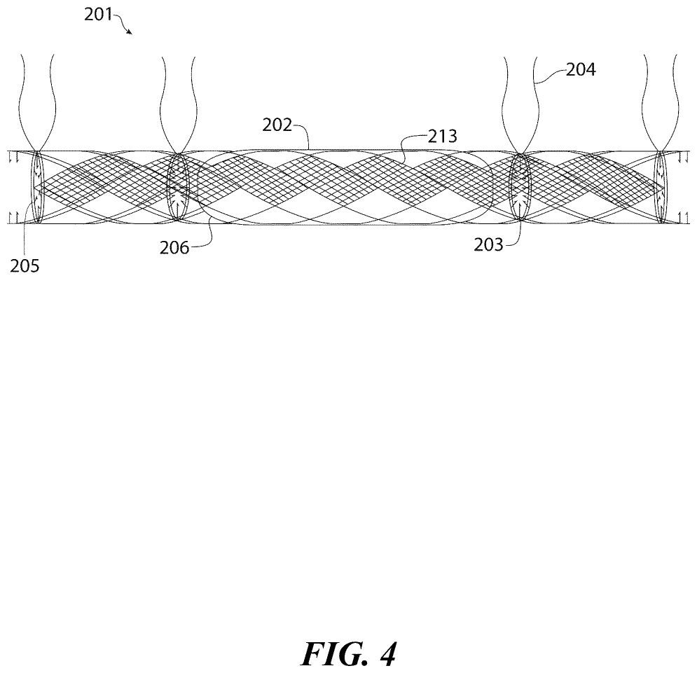

FIGS. 4-6 illustrate a second particular, non-limiting embodiment of a fibrous connective tissue stabilization apparatus in accordance with the teachings herein. With reference thereto, the fibrous connective tissue stabilization apparatus 201 includes a fibrous, tubular body 202 with a plurality of synchs 203 disposed therein. Each of the plurality of synchs 203 is equipped with external threads 204 which may be utilized to tighten the synchs 203 and reduce the diameter thereof. As in the embodiment of FIGS. 1-2, the synchs 203 in this embodiment are preferably equipped with a plurality of inward facing barbs 205 that serve the same or similar function as the inward facing barbs 105 of FIGS. 1-2. In addition, the fibrous connective tissue stabilization apparatus 201 in this embodiment is equipped with a repositionable fastener 213 (which may be, for example, a hook-and-loop type fastener such as that sold under the tradename VELCROR).

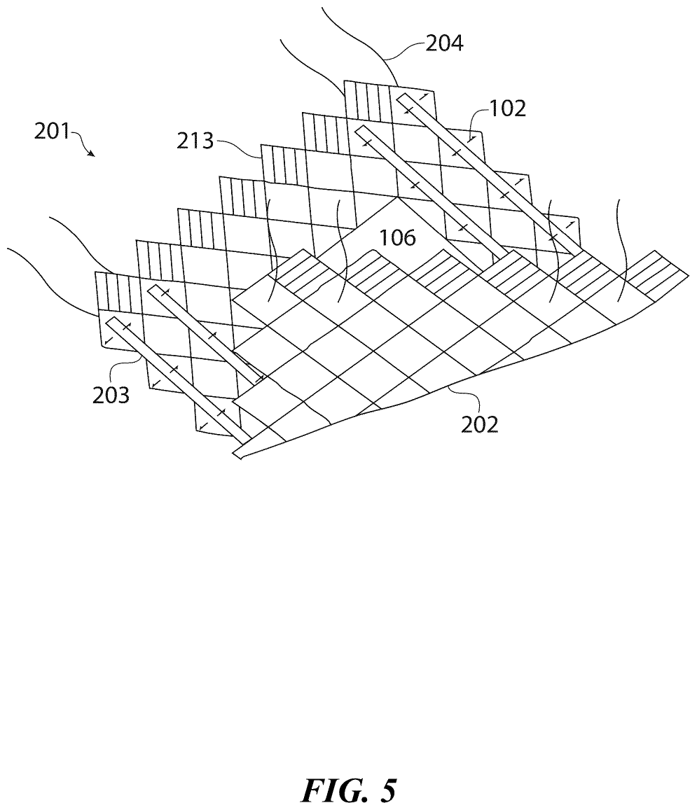

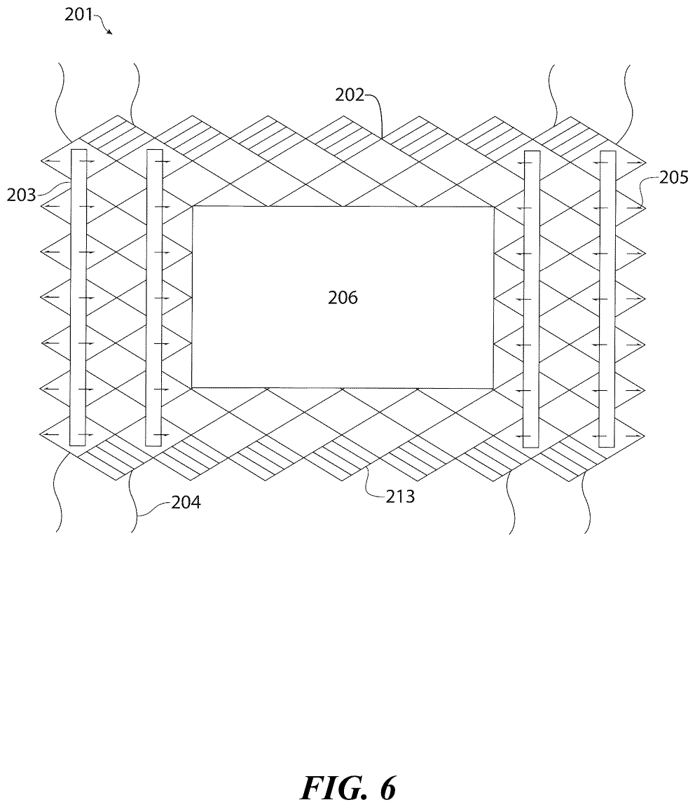

The utilization of this repositionable fastener 213 may be appreciated with respect to FIGS. 5-6. With reference thereto, the fibrous, tubular body 202 in this particular embodiment may be initially supplied as a flattened sheet (see FIGS. 5-6). This sheet may then be arranged around a frayed or damaged portion of connective tissue, after which adjacent ends of the sheet may be furled towards each other as shown in FIG. 5. The adjacent ends of the sheet may then be secured to each other by way of the repositionable fastener 213 to result in the tubular construct of FIG. 4, which will encompass the frayed or damaged portion of connective tissue.

Various materials may be utilized in the construction of the fibrous connective tissue stabilization apparatus disclosed herein. Preferably, these materials are biocompatible, and even more preferably, are bioresorbable or biodegradable. These materials include, but are not limited to, silk, cotton, biodegradable polyacrylic acid (polyHEMA/pDMA), poly(L-lactide) (PLLA), tyrosine poly carbonate and salicylic acid.

The fibrous connective tissue stabilization apparatus disclosed herein may assume various geometries and dimensions. The dimensions suitable for a particular application may depend on various factors including, for example, the location of the connective tissue to be stabilized, the extent to which the connective tissue has been frayed, and the age and weight of the patient.

Various additions and modifications may be made to the foregoing embodiments without departing from the scope of the present disclosure.

For example, some embodiments of the fibrous connective tissue stabilization apparatus disclosed herein may be provided with drawstrings equipped with adjustable or variable barb designs. Such embodiments may enable surgeons to tailor the engagement mechanism of the device to the specific requirements of the injured tissue, thereby ensuring optimal support and facilitating effective healing.

One important aspect of such embodiments is the capability to adjust the length and shape of the barbs on the drawstrings. This adaptability may ensure that the apparatus can be customized in real-time, allowing for precision engagement with the tissue based on its type and the severity of the injury. Such adjustable features may be designed to significantly enhance the versatility and effectiveness of the device across a broad spectrum of surgical scenarios, thereby improving overall tissue engagement and healing outcomes.

Some of the foregoing embodiments may feature mechanically adjustable barbs. These barbs may be engineered to extend or retract mechanically, granting surgeons the ability to modify their length in response to the varying densities and tear severities of the tissue encountered during surgery. This feature may provide precise control over the ability of the device to engage with tissues, thereby ensuring optimal stability and support.

Some possible embodiments with adjustable barb length and shape may use a combination of mechanically adjustable elements and shape-memory materials. For example, barbs made from a shape-memory alloy like Nitinol may be designed to change their shape or extend/retract in response to temperature changes or mechanical manipulation. This allows for the customization of barb engagement depending on the specific needs of the tissue being repaired, providing improved or optimal anchorage and support while reducing tissue trauma. The adjustment mechanism may be controlled externally or designed to respond autonomously to changes in the surgical environment.

Some of the foregoing embodiments may feature shape memory alloys. Incorporation of shape memory alloys into the barb design allows for substantial customization. For example, these materials may alter their shape in reaction to suitable stimuli (such as, for example, temperature changes or electrical stimuli), thereby offering surgeons the ability to adjust the barb shape to meet the unique demands of the surgical site.

For example, an embodiment using shape memory alloys might involve a device made from Nitinol, an alloy known for its shape-memory and superelastic properties. In such embodiments, the device may be compressed or deformed for minimal-invasive insertion. Once placed in the body and exposed to body temperature, it may revert to its predetermined shape, fitting the injury site more optimally or even perfectly. This property allows for a less invasive insertion process and a customized fit that adapts to the injury site, improving stabilization and supporting the healing process. The biocompatibility and resilience of Nitinol make it a promising material for such medical applications.

Some of the foregoing embodiments may feature variable barb profiles. Since different injuries require different engagement mechanisms, these embodiments feature barbs with a variety of profiles, including spiral, hooked, or straight. This diversity enables the selection and modification of the barb profile to achieve the best possible tissue engagement for each specific case.

For example, an embodiment with variable barb profiles may involve a device with barbs that can be modified in shape or size. For instance, using materials like stainless steel or titanium, the barbs may be designed to switch between different profiles (e.g., straight, hooked, or spiral) to optimize tissue engagement. This may be achieved through mechanical means (as, for example, through the provision of a sliding mechanism controlled during surgery), or by employing shape-memory materials that alter their form in response to stimuli (such as, for example, temperature or magnetic fields), providing a custom fit for various tissue types and repair needs.

Some of the foregoing embodiments may feature barbs having remote adjustment capability. A significant feature of this design is the ability to adjust the length and shape of the barbs remotely, and post-surgery. This non-invasive capability allows for adjustments to be made as the healing process advances, ensuring continued optimal support and adaptation to the changing needs of the healing tissues.

For example, an embodiment with remote adjustment capability may feature a device integrated with a microelectromechanical system (MEMS). This system may allow for the remote control of device features, such as the extension or retraction of barbs, using external magnetic or electric fields. The device may be made from materials like Nitinol or other responsive alloys, with the MEMS components being safely encapsulated in biocompatible materials. Such a setup may enable post-operative adjustments to the device without additional invasive procedures, enhancing patient comfort and recovery outcomes.

Some of the foregoing embodiments may feature biodegradable and morphing barbs. Utilizing biodegradable materials that morph in shape as they degrade adds another layer of dynamic support. This approach may help ensure that the apparatus adapts over time to the healing tissue, thus providing sustained engagement and support tailored to the evolving requirements of the tissue.

For example, embodiments equipped with biodegradable and morphing barbs may use a combination of biodegradable polymers and shape-memory materials. The barbs, made from a biodegradable polymer like polylactic acid (PLA), may be embedded with shape-memory alloy particles. This design may allow the barbs to change shape or gradually degrade in response to the healing process, adapting their form and mechanical properties to provide optimal support. As the tissue heals, the barbs may slowly dissolve, minimizing the need for surgical removal and reducing long-term tissue irritation.

To further enhance the surgical efficacy and patient safety associated with the fibrous connective tissue stabilization apparatus, some embodiments may feature retractable barbs. This feature is designed to significantly improve the versatility and safety of the device, facilitating smoother insertion into tissue and ensuring safer removal when necessary, thereby minimizing trauma and optimizing tissue engagement throughout the surgical process.

The disclosure herein of drawstrings equipped with retractable barbs represents a pivotal advancement in surgical device technology. This feature enables the barbs to be seamlessly retracted into the device's body or extended on demand, thereby offering enhanced control over the device's interaction with tissue at every stage of the surgical and healing process.

Some embodiments of the fibrous connective tissue stabilization apparatus may be equipped with a mechanism for retraction and extension. Such embodiments may employ a sophisticated mechanism that allows for the smooth retraction of barbs into the device's body, ensuring ease of insertion into the tissue. Once positioned, the barbs may be extended to securely engage with the tissue, providing the necessary stability and support for effective healing. This mechanism is preferably designed to be highly reliable and responsive, allowing for precise control over the device's deployment and engagement with the tissue.

One possible embodiment of such a device may be realized by using a small, integrated mechanical system made from biocompatible materials such as, for example, stainless steel or titanium. The system may be activated manually during surgery or through suitable remote activation mechanisms. Such a design may allow for precise control over the engagement of the barbs with the tissue, enhancing the device's adaptability and reducing tissue trauma during insertion and removal.

In the context of the fibrous connective tissue stabilization apparatus disclosed herein, the integration of a mechanism for the retraction and extension of barbs represents a significant advancement in surgical device technology. This mechanism enhances the device's adaptability, precision, and safety, minimizing tissue trauma during insertion and removal. The activation of this mechanism may be achieved through various innovative methods, both manually during surgery and remotely, offering flexibility and control in different surgical scenarios.

Manual activation during surgery may be facilitated through mechanical levers or sliders integrated into the device's handle. This approach allows surgeons to intuitively adjust the barbs' extension or retraction with simple hand movements, ensuring immediate response and precise control over the device's engagement with the tissue. Alternatively, a screw mechanism may be employed, where rotating a dial or screw on the device causes the barbs to extend or retract. This method provides granular control, enabling surgeons to make precise adjustments to the barb positioning. Additionally, a hydraulic or pneumatic system may be implemented, utilizing a handheld pump or bulb to control barb movement through fluid pressure. This offers a tactile and responsive method of activation, allowing for quick adjustments during surgery.

For remote activation, several possible methods may be utilized to extend or retract the barbs without direct manual intervention. A wireless control panel or remote may enable surgeons or their assistants to control the barb mechanism from a distance, enhancing flexibility in maintaining sterile fields and minimizing physical disruptions. Integration with a smartphone or tablet application offers a modern and versatile solution, providing a digital platform for device control, including barb retraction and extension. This method may also allow for monitoring and adjusting other device settings in real-time. Voice activation technology presents a hands-free option, enabling surgeons to command the device using voice commands, thus maintaining sterility and focusing on the surgical procedure without manual distractions. Lastly, a foot pedal connected to the device may offer another means of remote activation, allowing surgeons to use their foot to control the mechanism while keeping their hands free for other tasks.

These manual and remote activation methods underscore the innovative design and functionality of the fibrous connective tissue stabilization apparatus, significantly enhancing its utility in surgical settings. By offering various ways to control the retraction and extension of barbs, the device adapts to the surgeon's needs, improving the precision of tissue engagement and reducing trauma, thereby contributing to better surgical outcomes and patient recovery.

Some embodiments of the fibrous connective tissue stabilization apparatus disclosed herein may feature controlled deployment of barbs. Such embodiments may allow the surgeon to selectively engage them as needed, thereby reducing tissue trauma during insertion. For example, possible embodiments equipped with controlled deployment features may be equipped with a precision mechanical system made from biocompatible materials such as, for example, surgical-grade stainless steel. The mechanism may allow the surgeon to deploy the barbs gradually, ensuring precise placement and minimizing tissue trauma. Controlled deployment may be manually operated or may use a remotely activated system, offering flexibility and precision during the surgical procedure.

Some embodiments of the fibrous connective tissue stabilization apparatus disclosed herein may feature post-operative adjustment. Such embodiments offer the ability to retract the barbs post-operation, either for adjustment purposes or for eventual removal of the device, thereby minimizing tissue damage.

Possible embodiments with post-operative adjustment capabilities may involve devices equipped with a remotely controlled adjustment mechanism. This mechanism, which may employ smart materials (such as, for example, shape-memory alloys or piezoelectric materials), may be activated externally via a controlled energy source (such as, for example, magnetic fields or low-intensity ultrasound). The design may allow for non-invasive post-operative adjustments to the device's position, tension, or other features, providing tailored support as the patient's healing progresses. This approach may enhances patient comfort and recovery by reducing the need for additional surgical interventions.

Some embodiments of the fibrous connective tissue stabilization apparatus disclosed herein may feature an automated feedback system. In such embodiments, the automated feedback system may retract or extend the barbs in response to changes in tissue density or tension, thereby facilitating or optimizing engagement and support continuously.

Possible embodiments equipped with an automated feedback system may include devices integrated with smart sensors capable of monitoring tissue health, strain, or other relevant parameters. These sensors, which may comprise biocompatible materials (such as, for example, silicone or polyurethane), may provide real-time data on the healing process. The device may then automatically adjust its position, tension, or drug delivery in response to this data, using a mechanism such as, for example, a miniaturized motor or shape-memory alloy actuator. This system may improve or optimize the healing process by continuously adapting to the patient's recovery needs.

Some embodiments of the fibrous connective tissue stabilization apparatus disclosed herein may feature the use of biocompatible materials for the actuation mechanism to ensure safety and minimize irritation to the surrounding tissues. Such embodiments may involve, for example, a device using materials such as medical-grade silicone or biocompatible polymers for its actuation system. These materials may be designed to respond to stimuli such as temperature, pH, or mechanical forces to adjust the device's features (such as, for example, barb extension or retraction). The actuation may be achieved through material expansion or contraction properties or embedded micro-mechanisms. Such materials may ensure safety and compatibility with body tissues, thereby reducing the risk of adverse reactions while providing the necessary functional adjustments.

Some embodiments of the fibrous connective tissue stabilization apparatus disclosed herein may utilize different materials for the barbs, ranging from rigid to flexible, to match the varying requirements of different surgical situations. For example, more flexible barbs may be utilized for delicate tissues, while rigid barbs may be utilized for more robust support in severe tears. This concept aims to tailor the material properties of the barbs to the specific requirements of the surgical situation, thereby enhancing the efficacy and biocompatibility of the device.

For example, some embodiments of the fibrous connective tissue stabilization apparatus disclosed herein may utilize flexible polymers for barbs to engage delicately with sensitive tissues, reducing the risk of further damage. Such embodiments may utilize materials such as medical-grade silicone or polyurethane. These polymers may be shaped into barbs or other components of the device. Their flexibility ensures minimal tissue damage and discomfort upon insertion and during the healing process. The polymers may be designed to flex or bend in response to tissue movement, providing gentle but effective support. This adaptability may make them ideal for dynamic environments such as joint areas, where movement is constant, ensuring the device remains effective without causing additional stress to the healing tissue.

Some embodiments of the fibrous connective tissue stabilization apparatus disclosed herein may utilize rigid metals or alloys for barbs when stronger tissue engagement is needed, especially in cases of severe tears or where more robust anchoring is required. Such embodiments may involve materials such as titanium or stainless steel. These materials can be used to create barbs or anchors that require strong, non-flexible support, especially in severe tissue tears where robust anchoring may be crucial. The rigidity of these metals ensures they maintain their shape and provide consistent support. Suitable surgical procedures may involve embedding these metal components into the device, ensuring they are precisely positioned to offer maximum support and stability to the healing tissue. These metals may be chosen for their strength, biocompatibility, and resistance to corrosion, making them safe and effective for medical use.

Some embodiments of the fibrous connective tissue stabilization apparatus disclosed herein may utilize bioactive materials for the barbs that may promote tissue growth or healing, thereby enhancing the overall healing process. Such embodiments may involve coating or integrating materials such as hydroxyapatite or bioglass into the device. These materials are known for their ability to promote bone and tissue growth, making them desirable for enhancing healing. The bioactive component may be applied as a coating on the surface of the device or may be embedded within its structure. Upon implantation, these materials interact with the body's natural processes, encouraging tissue regeneration and integration, thereby accelerating the healing process and improving the overall effectiveness of the device.

Some embodiments of the fibrous connective tissue stabilization apparatus disclosed herein may utilize composite materials that combine flexibility with strength, providing an optimal balance for tissue engagement and support. Such embodiments may involve combining biodegradable polymers such as polylactic acid (PLA) with reinforcing materials such as carbon fiber or bioactive glass. This combination may create a composite that balances biodegradability with the necessary mechanical strength. The composite material may be used to construct parts of the device that require durability and support. The manufacturing process may involve techniques such as layering or blending these materials to achieve the desired properties, ensuring the device is strong and yet safely degrades over time as the tissue heals.

Some embodiments of the fibrous connective tissue stabilization apparatus disclosed herein may incorporate different surface textures on the barbs to modify the interaction with tissue and to promote better integration or grip. Such embodiments may utilize etching or laser patterning techniques on materials such as titanium or biocompatible polymers. Such processes may be utilized to create different surface textures (for example, smooth, rough, or grooved) on the device, thereby enhancing tissue grip and integration. The variation in texture may be tailored to specific parts of the device, thereby improving or optimizing interaction with various tissue types and improving the device's overall effectiveness in stabilizing and supporting the healing tissue.

Some embodiments of the fibrous connective tissue stabilization apparatus disclosed herein may incorporate biologically active coatings or materials in the barbs (such as, for example, materials which promote cell growth or anti-inflammatory agents) to aid in the healing process. This approach may be leveraged to add therapeutic functionality to the barbs, thereby contributing to a more efficient and effective healing process.

For example, some embodiments of the fibrous connective tissue stabilization apparatus disclosed herein may incorporate drug eluting barbs that release therapeutic agents (such as, for example, anti-inflammatory drugs or antibiotics), thereby aiding in reducing post-surgical complications and enhancing healing. Such embodiments may involve the use of biocompatible materials such as medical-grade polymers or metals for the barbs, coated with a layer of micro-encapsulated drugs. These drugs may be anti-inflammatory agents, antibiotics, or growth factors, and may be gradually released as the coating dissolves or is absorbed by the body. The barbs may be designed to engage the tissue, and as they do so, directly deliver the drugs to the target area, thereby enhancing healing and reducing the risk of infection. The manufacturing process may involve advanced techniques such as dip-coating or layer-by-layer assembly to ensure a consistent and effective drug coating on the barbs.

As another example, some embodiments of the fibrous connective tissue stabilization apparatus disclosed herein may utilize infusion of the barbs with growth factors to promote tissue regeneration and faster healing of the repaired site.

Such embodiments may use biocompatible materials (such as, for example, hydrogels or medical-grade polymers) for the device, infused with growth factors. These factors, which may be crucial for tissue regeneration, may be released gradually at the repair site. The manufacturing process may involve embedding or encapsulating the growth factors within the material matrix, thereby ensuring their stability and controlled release. This approach aims to enhance the healing process by directly providing essential biochemical cues to the damaged tissue.

As a further example, some embodiments of the fibrous connective tissue stabilization apparatus disclosed herein may involve application of a bioactive coating to the barbs that may interact positively with the surrounding tissue, thereby encouraging integration and reducing the chances of rejection. Such embodiments may involve, for example, applying a layer of bioactive substances (such as, for example, hydroxyapatite, collagen, or antibacterial agents) to the surface of the device. This coating, which may be applied through suitable processes such as, for example, dip-coating, spraying, or electrospinning, may interact with the body's tissues to promote healing, reduce inflammation, or prevent infection. Such coatings may be designed to gradually degrade, releasing their bioactive compounds over time to enhance the healing process effectively and safely.

As yet another example, some embodiments of the fibrous connective tissue stabilization apparatus disclosed herein may incorporate antimicrobial materials or coatings to prevent infection at the surgical site. Such embodiments may involve, for example, coating the device with antimicrobial agents such as, for example, silver nanoparticles, copper, or zinc oxide. These materials may be applied to the device through processes such as ion plating, dipping, or electrospinning. They provide a surface that actively inhibits bacterial growth, reducing the risk of infection post-surgery. This coating may be thin yet effective, ensuring the device remains biocompatible and functional while offering continuous antimicrobial protection.

In some embodiments of the fibrous connective tissue stabilization apparatus disclosed herein, the barbs may be embedded with micro-sensors to monitor stress, strain, or tissue integration. This may provide valuable feedback on the healing process and alert healthcare providers if any adjustments are needed. This modification may provide real-time, critical information on the healing process, thereby enhancing patient care and recovery outcomes.

For example, in some embodiments of the fibrous connective tissue stabilization apparatus disclosed herein, biocompatible microsensors may be embedded within the device. These sensors, which may comprise materials such as silicon or flexible polymers, may detect changes in tissue adherence, pressure, or biochemical markers indicating successful integration. Data from these sensors may be wirelessly transmitted for analysis, thereby providing real-time feedback on the healing process. Embodiments of this type potentially allow for non-invasive monitoring and timely intervention if issues arise, ensuring optimal healing and reducing the risk of complications.

As a further example, some embodiments of the fibrous connective tissue stabilization apparatus disclosed herein may incorporate sensors that measure the mechanical stress and strain on the barbs, thereby offering insights into the forces at the repair site. Some such embodiments may involve the integration of miniature strain gauges or piezoelectric sensors into the device. These sensors, which may comprise materials such as thin-film metal or piezoelectric ceramics, may measure the mechanical forces exerted on the device, thus indicating how well the tissue is healing and adapting to the repair. Data from these sensors may be wirelessly transmitted to a monitoring system, thereby providing valuable insights into the healing process and enabling timely adjustments to the treatment plan.

As still another example, some embodiments of the fibrous connective tissue stabilization apparatus disclosed herein may include sensors to detect temperature and pH changes, which may be early indicators of infection or inflammation. Such embodiments may incorporate microsensors made from materials such as conductive polymers or nanomaterials into the device. These sensors may be capable of detecting changes in temperature and pH levels, which are frequent indicators of inflammation or infection. Data from these sensors may be wirelessly transmitted for monitoring, enabling early detection of complications and facilitating timely medical interventions, thereby improving the chances of successful healing and reducing the risk of post-operative complications.

As yet another example, some embodiments of the fibrous connective tissue stabilization apparatus disclosed herein may feature barbs which are equipped with wireless capabilities to transmit data to external monitoring devices, thereby enabling non-invasive tracking of the healing progress. Such embodiments may involve integrating small wireless transmitters, such as RFID chips or Bluetooth-enabled microchips, into the device. These chips, which may be made from biocompatible materials, may collect data from the device's sensors and transmit it wirelessly to an external receiver. Such a setup may allow for real-time monitoring of critical parameters such as strain, temperature, or pH, thus facilitating timely medical responses to changes in the patient's condition, thereby enhancing the effectiveness of the treatment and recovery process.

Some embodiments of the fibrous connective tissue stabilization apparatus disclosed herein may entail the integration of a programmable system that allows the controlled release of drugs based on a predetermined schedule or in response to remote activation. Such embodiments may be employed to provide highly customizable and responsive drug delivery, thereby enhancing treatment efficacy and patient convenience.

For example, in some embodiments, the fibrous connective tissue stabilization apparatus disclosed herein may be equipped with a smart system that may be programmed to release drugs at specific times or in response to certain triggers.

As a further example, in some embodiments, the fibrous connective tissue stabilization apparatus disclosed herein may be equipped with remote control capabilities to allow healthcare providers to adjust the drug delivery schedule remotely, thereby adapting to the patient's changing needs without invasive procedures.

As yet another example, in some embodiments, the fibrous connective tissue stabilization apparatus disclosed herein may be equipped with real-time monitoring and adjustment capabilities. For example, such embodiments may integrate sensors that monitor the healing process and automatically adjust drug delivery in real-time based on the data collected.

Some embodiments of the fibrous connective tissue stabilization apparatus disclosed herein may utilize different biodegradable materials that can dissolve after a certain period, eliminating the need for surgical removal. This approach may align the device's support lifespan with the patient's specific healing needs, thereby minimizing or eliminating the need for surgical removal.

For example, some embodiments of the fibrous connective tissue stabilization apparatus disclosed herein may utilize materials with varied degradation times to tailor the device's longevity based on the estimated healing duration for different types of tissue injuries. For example, such embodiments may use layered biodegradable materials (such as, for example, PLA, PGA, or PCL (polycaprolactone)), each with distinct degradation rates. These layers may degrade sequentially, providing support that adapts over time. The device's structure may be designed such that the outer layers degrade faster, gradually transferring support to inner, slower-degrading layers. This approach may be utilized to align the device's functional lifespan with the tissue healing process. Manufacturing may involve techniques such as co-extrusion or layer-by-layer fabrication to create this multi-layered, biodegradable structure.

Some embodiments of the fibrous connective tissue stabilization apparatus disclosed herein may utilize a variety of biodegradable materials that degrade at different rates, tailored to match the healing timeline of various tissues or stages of recovery. In one such embodiment, the device may use a combination of biodegradable materials such as polydioxanone (PDO) for short-term support, and poly(L-lactic acid) (PLLA) for longer-term stability, both of which are known for their biocompatibility and different degradation rates. The specific choice of materials may be based on the required strength, flexibility, and degradation timeline matching the healing process. Advanced manufacturing techniques such as 3D printing or injection molding may be used to create the device, ensuring precision and adherence to medical standards.

Some embodiments of the fibrous connective tissue stabilization apparatus disclosed herein may utilize a layered structure. In such embodiments, the device may be equipped with multiple layers, each made of different biodegradable materials, so that each layer degrades sequentially, providing a phased reduction in support. In one such embodiment, the device may be constructed using multiple layers of biodegradable materials such as, for example, PLA (polylactic acid), PGA (polyglycolic acid), and PCL (polycaprolactone). Each layer may have a different degradation rate, tailored to match various stages of the healing process. The outer layers may be designed to degrade faster for immediate support, while inner layers provide longer-term stability. This may be achieved through advanced manufacturing techniques such as sequential 3D printing or layered casting, thereby ensuring precise control over the thickness and composition of each layer.

Some embodiments of the fibrous connective tissue stabilization apparatus disclosed herein may be designed to provide customized healing support. For example, in such embodiments, the degradation rates may be tailored to provide initial robust support immediately post-surgery, which gradually reduces as the natural healing process takes over. Such embodiments may utilize smart materials such as hydrogels or biodegradable polymers which are programmed to change their properties over time. These materials may be engineered to gradually soften, allowing for initially rigid support that becomes more flexible as the tissue heals. This customization may be achieved by blending materials with different degradation rates or incorporating responsive elements that react to physiological changes. The manufacturing process may involve advanced techniques such as, for example, multi-material 3D printing or sequential layering, thereby ensuring precise control over the material properties throughout the device.

Some embodiments of the fibrous connective tissue stabilization apparatus disclosed herein may feature composite materials that combine different biodegradable polymers, offering a balance of strength and flexibility, and allowing for a gradual transfer of load from the device to the healing tissue. This approach may be utilized to create a more versatile and effective biodegradable material that may be tailored to specific medical situations and patient needs. For example, such embodiments may use a blend of PLA (polylactic acid) and PGA (polyglycolic acid), possibly enhanced with bioactive components such as hydroxyapatite for bone integration. These materials may be combined using co-extrusion or layered fabrication techniques. The composite structure may offer both strength and controlled degradation, which may be tailored to the healing timeline. The bioactive enhancement may promotes tissue growth and integration, making the device effective for bone and soft tissue repair. This approach combines the benefits of different materials for optimal healing support.

Some embodiments of the fibrous connective tissue stabilization apparatus disclosed herein may combine different biodegradable polymers, each with unique degradation rates and mechanical properties, to create a material that offers optimal support and degrades appropriately as tissues heal. For example, such an embodiment may utilize a composite of biodegradable polymers such as PLA and PCL, blended with bioactive materials such as hydroxyapatite or collagen. This hybrid material may be fabricated using techniques such as electrospinning or 3D printing, thereby creating a structure that combines mechanical strength with biological activity. The bioactive components may enhance tissue integration and healing, while the polymers provide structural support that gradually degrades. This approach may be utilized to create a scaffold that supports the healing process while being gradually replaced by natural tissue.

Some embodiments of the fibrous connective tissue stabilization apparatus disclosed herein may be fabricated with tailored degradation profiles. In such embodiments, the composite materials may be engineered to have specific degradation profiles, allowing for parts of the device to degrade faster than others, depending on the required support and healing timeline. Such embodiments may utilize a combination of biodegradable polymers (such as, for example, PLA, PGA, and PCL), each with different degradation rates. These materials may be layered or blended using techniques such as co-extrusion or additive manufacturing. The design of these embodiments may allow for a controlled degradation that matches the healing timeline, for example by providing initial robust support that gradually reduces as the body heals. A tailored degradation profile of this type may be utilized to ensure that the device's structural integrity aligns with the tissue's recovery needs.

Some embodiments of the fibrous connective tissue stabilization apparatus disclosed herein may utilize distinct materials of different strength to enhance overall properties (such as, for example, tensile strength, flexibility, or biocompatibility), thereby providing a more effective and patient-specific healing environment. For example, such embodiments may use a composite material combining biodegradable polymers (such as, for example, PLA or PGA) with reinforcing fibers (such as, for example, carbon or glass fibers). This approach may be utilized to create a material with increased strength and resilience which is suitable for load-bearing applications. The manufacturing process may involve techniques such as filament winding or 3D printing, thus allowing precise control over fiber orientation and distribution, and thereby improving or optimizing the mechanical properties of the device to suit specific medical needs.

Some embodiments of the fibrous connective tissue stabilization apparatus disclosed herein may utilize pH-sensitive materials that degrade in response to changes in pH level, which may correlate with different stages of the healing process or the presence of infection. This approach may be utilized to create a smart, responsive system that adapts to the healing environment, enhancing the effectiveness and safety of the treatment. For example, such embodiments may utilize polymers that respond to pH changes, such as poly(acrylic acid) or chitosan-based hydrogels. These materials may be used to coat or construct parts of the device, thus allowing them to change properties in response to the pH levels typical of healing tissues or infection sites. For example, these materials may swell, degrade, or release embedded drugs at specific pH levels. The manufacturing process for these embodiments may involve standard polymerization techniques, with careful calibration to ensure the desired pH responsiveness.

One specific embodiment t of the foregoing type may use biodegradable polymers such as polycaprolactone (PCL) or poly(lactic-co-glycolic acid) (PLGA), which are engineered to degrade at rates responsive to environmental conditions like pH or enzymatic activity. These materials may be processed through techniques such as injection molding or 3D printing to form the device, with specific design considerations to ensure that the degradation rate aligns with the healing process. This approach may allow the device to provide support as needed, and then gradually dissolve as the tissue recovers.

Some embodiments of the fibrous connective tissue stabilization apparatus disclosed herein may utilize materials that change properties in response to infection indicators, such as pH or the presence of specific enzymes. For example, such a device may incorporate a polymer such as chitosan, which is known for its antimicrobial properties, and which may be activated or degrade faster in the presence of infection. This responsive action may release antimicrobial agents or signal the need for medical intervention. The manufacture of such embodiments may involve incorporating these responsive elements into the device's structure, thereby ensuring they are beneficially or optimally placed for effective infection detection and response.

Some embodiments of the fibrous connective tissue stabilization apparatus disclosed herein may integrate pH-sensitive components into the drug delivery system, where the release of therapeutic agents is triggered by changes in the local pH, thereby providing targeted treatment in response to healing needs. Such embodiments may involve, for example, incorporating microencapsulated drugs within a biodegradable polymer matrix. Materials such as PLGA may be utilized to encapsulate drugs, ensuring a slow and controlled release as the polymer degrades. Such a system may be implemented by embedding these microcapsules into the device using techniques such as co-extrusion or layering during the manufacturing process. This approach allows the device to provide sustained, localized drug delivery, enhancing healing while reducing systemic side effects.

Some embodiments of the fibrous connective tissue stabilization apparatus disclosed herein may utilize temperature sensitive materials, and in particular, materials that degrade at body temperature but remain stable at lower temperatures, thereby facilitating easier storage and handling before implantation. This approach may be utilized to create a device that dynamically adjusts to the body's healing environment, enhancing comfort and effectiveness. Some such embodiments may include shape-memory polymers, thermo-responsive hydrogels or phase change materials (PCMs). Possible shape-memory polymers may include materials such as polyurethane-based shape-memory polymers, which change shape at specific temperatures. Such materials may be utilized for devices that need to expand or contract post-implantation.

Thermo-responsive hydrogels include hydrogels that swell or shrink in response to temperature changes. This feature may be utilized for drug release systems, where the hydrogel expands to release medication at body temperature. PCMs include PCMs such as paraffin wax encapsulated within the device, which melt and solidify at specific temperatures, and which may be used for controlled drug release or to provide thermal therapy.

Some embodiments of the fibrous connective tissue stabilization apparatus disclosed herein may utilize materials that change their physical properties, such as stiffness or elasticity, in response to body temperature variations, thereby allowing the device to adapt its support level as the healing tissue's needs change.

For example, such embodiments may include shape-memory alloys, such as Nitinol, which can change shape at body temperature, offering dynamic support adapted to the healing stage. Such embodiments may also include smart hydrogels, that is, hydrogels that adjust their stiffness or volume in response to temperature or pH changes, providing varying support levels as the body heals. These materials may be designed and processed (for example, via molding or 3D printing) to match specific medical requirements, such as the desired adaptation rate or mechanical strength. The foregoing approaches and materials may be utilized to create a device that provides optimal support throughout different stages of healing.

Some embodiments of the fibrous connective tissue stabilization apparatus disclosed herein may i8ncorporate materials that begin to degrade when they reach a certain temperature range, thereby aligning the device's lifespan with the healing stages that typically correspond to certain temperature profiles. Possible embodiments of this type may be fabricated with, for example, thermo-responsive polymers (for example, polymers such as poly(N-isopropylacrylamide)) that degrade or change properties at specific temperatures. Some such embodiments may also include biodegradable phase change materials, that is, materials that degrade when they reach a certain temperature range during the body's healing process. Such materials may be incorporated into the device's construction through suitable techniques such as injection molding or 3D printing, thereby ensuring, for example, that they degrade appropriately in response to the body temperature changes that align with healing stages.

The above description of the present invention is illustrative, and is not intended to be limiting. It will thus be appreciated that various additions, substitutions and modifications may be made to the above described embodiments without departing from the scope of the present invention. Accordingly, the scope of the present invention should be construed in reference to the appended claims.

What is claimed is:

A1. A method for repairing a torn portion of fibrous connective tissue comprising:
placing the torn portion of the fibrous connective tissue within a tubular construct consisting of a biocompatible, surgically implantable material, wherein the tubular construct is equipped with at least one barbed drawstring having a plurality of barbs on a surface thereof, and wherein the drawstring transforms the tubular construct from a first configuration in which the tubular construct has a first minimum diameter, to a second configuration in which the tubular construct has a second minimum diameter that is less than the first minimum diameter; and
using the drawstring to transform the tubular construct into the second configuration;
wherein, when the tubular construct is in the second configuration, the plurality of barbs engage the fibrous connective tissue.

A2. The method of claim 1, wherein the fibrous connective tissue is selected from the group consisting of tendons and ligaments.

A3. The method of claim 1, wherein the biocompatible, surgically implantable material Is selected from the group consisting of cotton and silk.

A4. The method of claim A1, wherein the at least one barbed drawstring comprises a biodegradable material selected from the group consisting of polyacrylic acid (polyHEMA/pDMA), poly(L-lactide) (PLLA), tyrosine polycarbonate, and salicylic acid.

A5. The method of claim A1, further comprising adjusting the length or shape of the barbs on the at least one barbed drawstring post-implantation, wherein the adjustment is performed non-invasively.

A6. The method of claim A1, wherein the at least one barbed drawstring includes barbs that are hollow and treated with one or more pharmaceutically active materials to facilitate the regrowth of the fibrous connective tissue by localized delivery of said materials.

A7. The method of claim A1, wherein the tubular construct transitions from the first configuration to the second configuration at least partially through the activation of a mechanism responsive to external stimuli selected from the group consisting of temperature, pH, and magnetic fields.

A8. The method of claim A1, wherein the tubular construct includes a plurality of barbed drawstrings distributed uniformly around its circumference.

A9. The method of claim A1, further comprising employing an automated feedback system integrated within the tubular construct to dynamically adjust the engagement of the barbs with the fibrous connective tissue in response to changes in at least one parameter selected from the group consisting of tissue density, tension, and healing status.

A10. The method of claim A1, wherein the barbs on the at least one barbed drawstring are fabricated with variable profiles selected from the group consisting of spiral, hooked, and straight profiles, to match the engagement needs of different types of fibrous connective tissue injuries.

A11. The method of claim A1, further comprising utilizing barbs fabricated from a shape-memory alloy that are adapted to adjust their engagement with the fibrous connective tissue in response to temperature changes.

A12. The method of claim A1, wherein the barbs on the at least one barbed drawstring are coated with a biologically active substance selected from the group consisting of hydroxyapatite, collagen, and antibacterial agents.

A13. The method of claim A1, further comprising a step of selecting the at least one barbed drawstring from a set with varying barb densities and configurations, allowing for the customization of the apparatus based on the severity and type of the fibrous connective tissue injury.

A14. The method of claim A1, wherein the tubular construct further comprises an outer layer treated with an antimicrobial coating.

A15. The method of claim A1, further comprising the use of a controlled release mechanism integrated within the barbs adapted to provide sustained delivery of therapeutic agents directly to the repair site over a predetermined period of time.

A16. The method of claim A1, wherein the tubular construct includes an adjustable closure mechanism adapted to facilitate the securement and adjustment of the fit of the tubular construct around the fibrous connective tissue post-implantation.

A17. The method of claim A1, further comprising embedding within the tubular construct a wireless sensor network capable of monitoring parameters indicative of healing progress, wherein said parameters are selected from the group consisting of pressure, tension, and biochemical markers, and wherein the data is transmitted to an external device.

A18. The method of claim A1, wherein the barbed drawstring includes a mechanism for mechanical actuation controlled via an external magnetic field to provide for the non-invasive adjustment of barb engagement with the tissue.

B1. A surgically repaired fibrous connective tissue, comprising:
first and second portions of torn fibrous connective tissue; and
a tubular construct encompassing torn portions of first and second portions of said torn fibrous connective tissue, said tubular construct consisting of a biocompatible, surgically implantable material, wherein the tubular construct is equipped with at least one barbed drawstring having a plurality of barbs on a surface thereof that engage the first and second portions of said torn fibrous connective tissue.

B2. The surgically repaired fibrous connective tissue of claim B1, wherein the biocompatible surgically implantable material of the tubular construct is selected from a group consisting of biodegradable polymers including poly(L-lactide) (PLLA), polyglycolic acid (PGA), and polycaprolactone (PCL).

B3. The surgically repaired fibrous connective tissue of claim B1, wherein the at least one barbed drawstring includes barbs that are hollow and capable of delivering pharmaceutically active materials directly to the site of the torn fibrous connective tissue.

B4. The surgically repaired fibrous connective tissue of claim B1, wherein the at least one barbed drawstring is equipped with a mechanism allowing for the remote adjustment of the barbs' protrusion or retraction to dynamically adapt to the healing tissue's needs without additional surgical intervention.

B5. The surgically repaired fibrous connective tissue of claim B1, wherein the tubular construct is equipped with an automated feedback system that adjusts the tension of the at least one barbed drawstring in response to changes in tissue healing.

B6. The surgically repaired fibrous connective tissue of claim B1, wherein the barbs on the at least one barbed drawstring are designed with variable profiles selected from the group consisting of spiral, hooked, and straight profiles.

B7. The surgically repaired fibrous connective tissue of claim B1, wherein the tubular construct encompasses a repositionable fastener mechanism which allows for adjustability and securement of the tubular construct around the torn portions of fibrous connective tissue.

B8. The surgically repaired fibrous connective tissue of claim B7, wherein the repositionable fastener mechanism is a hook-and-loop type fastener.

B9. The surgically repaired fibrous connective tissue of claim B1, further comprising embedded sensors disposed within the tubular construct which monitor the healing process in real-time, wherein said sensors providing data relating to healing indicators selected from the group consisting of tissue integration and strain.

B10. The surgically repaired fibrous connective tissue of claim B1, wherein the tubular construct is further characterized by an inner lining made from a hydrogel material that swells in response to body temperature to gradually releasing embedded, pharmaceutically active substances directly to the healing tissue.

B11. The surgically repaired fibrous connective tissue of claim B1, wherein the at least one barbed drawstring of the tubular construct is equipped with barbs fabricated from a shape-memory alloy.

B12. The surgically repaired fibrous connective tissue of claim B1, further comprising a mechanism within the tubular construct that emits low-level ultrasound waves that stimulating the cellular processes involved in tissue repair and accelerated the healing of the fibrous connective tissue.

B13. The surgically repaired fibrous connective tissue of claim B1, wherein the at least one barbed drawstring incorporates a mechanism for piezoelectrically induced drug delivery, allowing for the controlled release of therapeutics in response to mechanical pressure changes within the tissue environment.

B14. The surgically repaired fibrous connective tissue of claim B1, wherein the tubular construct is augmented with a biocompatible, electrically conductive mesh layer adapted to provide electrical stimulation to the repair site, thereby promoting tissue healing and regeneration through enhanced cellular activity.

B15. The surgically repaired fibrous connective tissue of claim B1, wherein the tubular construct includes an integrated sensor system capable of measuring and wirelessly transmitting data on at least one of tissue strain, oxygenation levels, and local temperature, enabling real-time monitoring of the healing environment.

B16. The surgically repaired fibrous connective tissue of claim B1, wherein the tubular construct is constructed from a composite material that combines biodegradable polymers with embedded nanofibers, thereby providing enhanced mechanical strength and controlled degradation rates tailored to match the healing timeline of the fibrous connective tissue.
